(19) United States
(12) Patent Application Publication
(10) Pub. No.: US 2004/0014107 A1
(43) Pub. Date: Jan. 22, 2004

(54) RNP immunoprecipitation assay

(76) Inventors: Mariano A. Garcia-Blanco, Durham, NC (US); Somashekarappa Niranjanakumari, Germantown, MD (US); Erika L. Lasda, Durham, NC (US); Robert M. Brazas, Middleton, WI (US)

Correspondence Address: NIXON & VANDERHYE, PC, 1100 N GLEBE ROAD, 8TH FLOOR, ARLINGTON, VA, 22201-4714, US

(21) Appl. No.: 10/443,129

(22) Filed: May 22, 2003

Related U.S. Application Data
(60) Provisional application No. 60/382,053, filed on May 22, 2002.

(51) Int. Cl. C12Q 1/68

(52) U.S. Cl. 435/6

(57) ABSTRACT

The present invention relates to a method of identifying an RNA, or specific region thereof, to which a protein of interest binds.

This application claims priority from Provisional Application No. 60/382,053, filed May 22, 2002, the entire content of which is incorporated herein by reference.

### TECHNICAL FIELD

The present invention relates to a method of identifying an RNA, or specific region thereof, to which a protein of interest binds.

### BACKGROUND

Ribonucleoprotein complexes (RNPs) are diverse macromolecular assemblies composed of both RNA and protein components. They play a number of essential roles in the maturation of most RNAs and in the translation of messenger RNAs (Varani and Nagai, Annu. Rev. Biophys. Biomol. Struct. 27:407-445 (1998), Cusack, Curr. Opinion in Struc. Biol. 9:66-73 (1999)). These processes are often temporally and spatially regulated within the cell by a network of RNA-protein and protein-protein interactions. To date, there are several in vitro and in vivo methods available to study RNA-protein interactions. In vitro techniques have been instrumental for the precise characterization of RNA and protein interaction sites. Methods such as filter binding and gel mobility shift assays are useful in measuring or approximating binding constants of RNA-protein complexes (Yarus and Berg, Anal. Biochem. 35:450-465 (1970), Setzer, Methods Mol. Biol. 118:115-128 (1999)). Enzymatic probing (Parker and Steitz, Methods Enzymol. 180:454-468 (1989)), hydroxyl radical footprinting (Huttenhofer and Noller, EMBO J. 13:3892-3901 (1994)), UV crosslinking (Dreyfuss et al, Mol. Cell Biol. 4:1104-1114 (1984)), photoaffinity crosslinking (Hanna et al, Methods Mol. Biol., 118:21-33 (1999)), and directed chemical nuclease probing (Joseph and Noller, Methods Enzymol. 318:175-190 (2000), Culver and Noller, Methods Enzymol. 318:461-475 (2000)), coupled with reverse transcription and peptide sequencing, have been effective in identifying the interacting components as well as in precisely pinpointing the site of RNA-protein interactions in vitro. RNA affinity tags (Srisawat and Engelke, RNA 7:632-641 (2001)) and RNA co-immunoprecipitation assays (Steitz, Methods Enzymol. 180:468-481 (1989)) are also useful for studying RNPs. Although the in vitro methods are valuable in understanding the structure/function relationship of RNP complexes, they may fail to reflect physiological interactions, which can be addressed only by in vivo assays.

In vivo assays such as the Tat-fusion assay (Landt et al, Methods Enzymol. 318:350-363 (2000), Cullen, Methods Enzymol. 328:322-329 (2000)), the frameshifting assay (Kollmus et al, RNA 2:316-323 (1996)), and the translational repression assay (Paraskeva et al, Proc. Natl. Acad. Sci. USA 95:951-956 (1998)) are used in the functional dissection of RNA-protein complexes in eukaryotes. Yeast three hybrid assays are powerful in identifying the binding partners in RNP complexes when one of the components is known (SenGupta et al, Proc. Natl. Acad. Sci. USA 93:8496-8501 (1996)). These methods, however, may fail to reveal native interactions. In vivo UV crosslinking has the advantage of analyzing RNA-protein interactions in their native architecture, however, it requires prolonged exposure permitting redistribution of components and the crosslinking of UV-damaged molecules (Dreyfuss et al, Mol. Cell Biol. 4:1104-1114 (1984)). Recently, RNP tagging has been used in combination with cDNA microarrays to identify mRNA subsets present in endogenous mRNP complexes (Tenenbaum et al, Proc. Natl. Acad. Sci. USA 97:14085-14090 (2000)). Although this approach is useful in understanding the endogenous mRNP complexes, redistribution of cellular components upon cell disruption or re-assortment of non-associated macromolecules during isolation are potential problems. When studying macromolecular interactions in vivo, it is essential to utilize methods that rapidly “freeze” these interactions as well as prevent the re-assortment of protein and RNA components during cell lysis. Crosslinking agents have been exploited for this purpose. In particular, crosslinking agents that are reversible are the most useful because they simplify subsequent characterization of the interacting molecules. Formaldehyde is a reversible crosslinking agent that has been used extensively as a fixative to preserve the structural integrity of cells. It has also been used in chromatin immunoprecipitation (ChIP) assays, which have proven to be a powerful method in delineating many DNA-protein interactions (Jackson, Cell 15:945-954 (1978), Orlando et al, Methods, Companion Methods Enzymol. 11:205-214 (1997), Orlando, Trends Biochem. Sci. 25:99-104 (2000), Shang et al, Cell 103:843-852 (2000)).

The present invention provides a RNP immunoprecipitation (RIP) assay utilizing formaldehyde as a reversible crosslinker to identify RNA-protein interactions in vivo.

### SUMMARY OF THE INVENTION

The present invention relates to a method of identifying an RNA, or specific region thereof, to which a protein of interest binds.

Objects and advantages of the present invention will be clear from the description that follows.

### BRIEF DESCRIPTION OF THE DRAWINGS

FIG. 1. Diagramatic representation of RIP assay using formaldehyde. Live cells in PBS are crosslinked with formaldehyde. Crosslinking is quenched by adding glycine and the fixed cells are lysed by sonication. A specific antibody against the target protein is used to immunoprecipitate crosslinked complexes, and the immunoprecipitates are washed with stringent buffers. The crosslinks are reversed, and the RNA is isolated and analyzed by RT-PCR using specific primers.

FIGS. 2A-2C. RIP assay using α-HDAg antibody specifically immunoprecipitates crosslinked HDV RNPs. (FIG. 2A) RT-PCR analysis of crosslinked, α-HDAg immunoprecipitated HDV RNPs. HEp-2 cells that were transfected with the wild type HDV expression plasmid were used for preparing uncrosslinked and crosslinked lysates for immunoprecipitations using α-HDAg antibody. After crosslinking, the isolated RNA samples were analyzed by RT-PCR using HDV-specific primers (926F and 1468R). +RT (upper panel) and −RT (lower panel) represent RT reactions carried out in the presence and absence of reverse transcriptase. The arrow indicates the amplified HDV-specific product. Lanes 1-2 and 7-8, input and supernatant from uncrosslinked and crosslinked samples respectively. Lanes 3-6 and 9-12, immunoprecipitated samples from uncrosslinked and crosslinked lysates respectively. 0 M, immunoprecipitated sample washed with RIPA buffer; 1 M, 2 M and 4 M, immunoprecipitated samples washed with high stringency RIPA buffer with the indicated urea concentration. Lanes 13-16 (−Ab), mock immunoprecipitated samples in the presence of protein-A Sepharose beads and absence of antibody. (FIG. 2B) RT-PCR analysis using U1-specific primers. The uncrosslinked and crosslinked α-HDAg immunoprecipitated samples from FIG. 2A were analyzed by RT-PCR using U1-F and U1-R primers that specifically amplify a 155 bp product from U1 snRNA. The lanes are as described in FIG. 2A. (FIG. 2C) RT-PCR analysis of the immunoprecipitated wild type and mutant samples. HEp-2 cells transfected with the wild type or the mutants, HDAg-255 and HDAg-257 (defective in producing HDAg) were crosslinked, immunoprecipitated with α-HDAg antibody and analyzed by RT-PCR using HDV-specific primers (926F and 1468R). Lanes 1, 4 and 7, input; lanes 2, 5 and 8 (−Ab), mock immunoprecipitated samples in the presence of protein-A Sepharose beads and the absence of antibody; and, lanes 3, 6 and 9, samples immunoprecipitated in the presence of α-HDAg antibody.

FIG. 3. Heterologous antibodies do not immunoprecipitate HDV RNPs. HEp-2 cells that were transfected with the HDV wild type expression plasmid were crosslinked and the lysates were immunoprecipitated with α-Sm antibodies and analyzed by RT-PCR using HDV-specific primers (926F and 1468R), which produce a 542 bp product. −RT (left panel, lanes 1-6) and +RT (right panel, lanes 7-12) represents RT reactions carried out in the presence and absence of reverse transcriptase. Lanes 1 and 7, input; lanes 2, 4, 8 and 10, supernatants from AW (+) and JM (−) immunoprecipitates; and, lanes 3, 5, 9 and 11, AW (+) and JM (−) immunoprecipitated samples, respectively. Lanes 6 and 12 (−Ab), mock immunoprecipitations in the absence of antibody.

FIG. 4. RIP assay using αα-Sm antibodies specifically immunoprecipitates crosslinked U1 snRNPs. RNA isolated from crosslinked, AW (+) or JM (−) immunoprecipitated samples was analyzed by RT-PCR using U1 and U3 snRNA-specific primers, U1-F and U1-R (upper panel) which produces a 155 bp U1-specific product and U3-F and U3-R (lower panel) which produces a 194 bp U3-specific product. −RT (left panel, lanes 1-6) and +RT (right panel, lanes 7-12) represents RT reactions carried out in the presence and absence of reverse transcriptase. Lanes 1 and 7, input samples; lanes 2, 4, 8 and 10, supernatants from AW (+) and JM (−) immunoprecipitates; and, lanes 3, 5, 9 and 11, AW (+) and JM (−) immunoprecipitated samples, respectively. Lanes 6 and 12 (−Ab), mock immunoprecipitations in the absence of antibody.

### DETAILED DESCRIPTION OF THE INVENTION

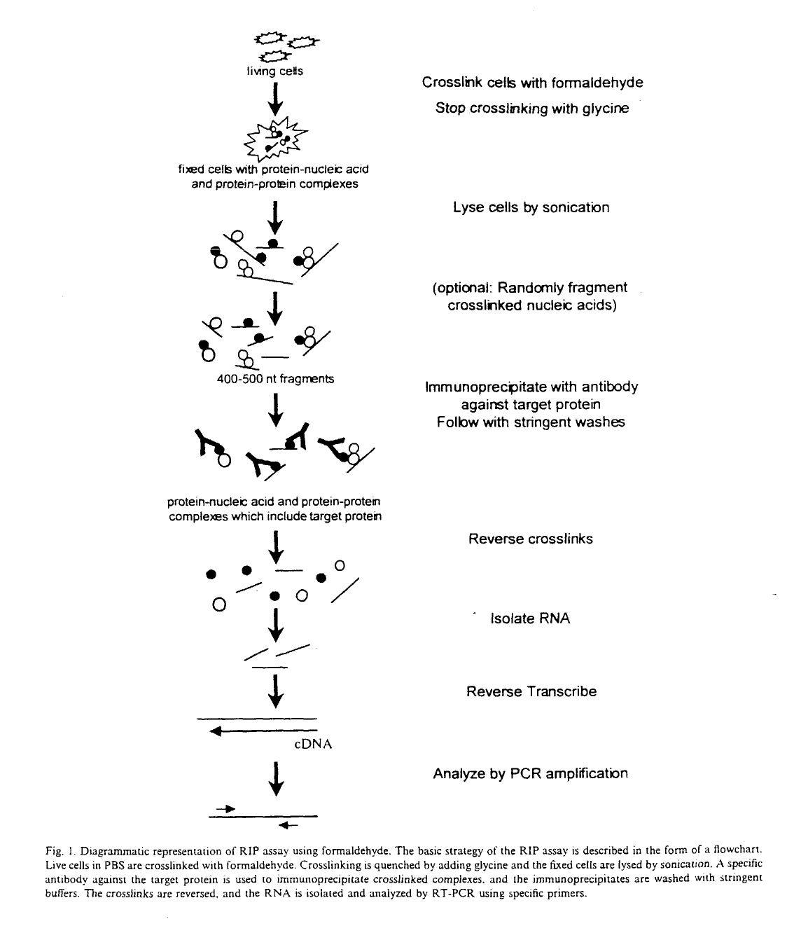

The present invention relates to a method of identifying RNA-protein interactions. The method comprises crosslinking cells with a reversible crosslinking agent, lysing the cells, immunoprecipitating from the lysate a target protein complexed to an RNA using an antibody specific for the target protein, reversing the crosslinks and analyzing the resulting RNA and/or protein. A basic outline of the assay is provided in FIG. 1.

More specifically, the invention relates to a method of identifying an RNA of a cell, or specific region thereof, to which a protein of interest (target protein) binds. In a first step, cells in tissue culture (suspension or monolayer), in a tissue ex vivo or in vivo in a living organism, are crosslinked using formaldehyde or other reversible crosslinker. Advantageously, formaldehyde is used at a final concentration of 0.1% (v/v) to 10% (v/v), preferably about 1% (v/v). In a preferred embodiment, the cells are incubated with the formaldehyde at about 25° C. for about 0.5 to 30 min, typically about 10 min, with gentle agitation. The use of a buffer containing primary or secondary amines is disadvantageous as formaldehyde can crosslink such reactive amines to proteins or nucleic acids. Phosphate, Hepes and triethanolamine buffers are preferred.

Optimization of the crosslinking conditions is desirable as excessive crosslinking can result in the loss of material due to poor solubilization or can mask epitopes recognized during subsequent immunoprecipitations. By contrast, suboptimal crosslinking can result in incomplete fixation resulting in fewer crosslinked complexes. Optimization can be carried out using, for example, formaldehyde concentrations from about 0.1 to about 1.0% and a duration of fixation from about 5 min to about 1 hour and the best combination for any given RNA-protein interaction determined.

Upon completion of the crosslinking step, the reaction can be quenched. Glycine or other appropriate compound with a primary amine can be used as the quencher when crosslinking is effected using formaldehyde. For example, glycine (e.g. pH 7) can be added to a final concentration roughly equal to that of the formaldehyde used, e.g., about 0.25M, and the resulting solution incubated briefly, e.g. about 5 min at room temperature.

The cells can then be harvested, e.g., by centrifugation, and washed, for example with cold PBS. Further processing can proceed immediately or the cells can be frozen and stored.

Fixed cells can be resuspended in a buffer, advantageously, one containing a protease inhibitor to prevent destruction of proteins, and treated to prevent unwanted RNA degradation by contaminating nucleases. Advantageously, the buffer has a pH less than 7.6 to avoid alkali hydrolysis of RNA. Detergents and EDTA can be included in the buffer to diminish non-specific interactions during processing of the crosslinked complexes. In certain cases, high salt (e,g., >1M NaCl, KCl or KOAc) and/or a denaturant (e.g., >1M urea) is added. An example of a suitable buffer is 0.50 mM Tric-Cl, pH 7.5, 1% Nonidet P40 (NP-40), 0.5% sodium deoxycholate, 0.5% SDS, 1 mM EDTA and 150 mM NaCl.

Fixed cells are highly rigid and thus resistant to detergent lysis or enzymatic digestion. As a result, solubilization is advantageously effected by mechanical shearing, for example, using sonication. The effect of shearing can be monitored microscopically. Remaining insoluble material can be removed, for example, by centrifugation.

A preclearing step can next be preformed to minimize the carry over of non-specific large aggregates that can increase background in subsequent steps. Preclearance can be effected by centrifugation or filtration.

When mapping of a specific binding site of a protein is desired, the crosslinked RNA sample can be fragmented to generate, for is example, fragments of about 200 or about 400-500 nucleotides in length. Fragmentation can be effected using, for example, sonication or limited nuclease digestion. However, when the only determination to be made is whether a protein interacts with a specific RNA, fragmentation can be omitted.

Crosslinked complexes (RNA-protein and protein-protein complexes) can be removed from the solution (solubilized cell lysate) resulting from the foregoing steps by immunoprecipitation using an antibody (e.g., polyclonal or monoclonal), or antigen binding fragment thereof, that binds (specifically) to the protein of interest. The antibody (or fragment thereof) can be bound to a solid support, e.g., a bead or membrane. For example, protein A or protein-G Sepharose beads coated with the antibody can be incubated with the lysate (e.g., precleared as described above) and then collected, for example, by centrifugation. The support-bound complexes can then be washed using a high stringency buffer (that is, a buffer that disrupts non-covalent interactions while leaving the crosslinked interations intact) (e.g., 50 mM-Tris-Cl, pH 7.5 1% NP-40, 1% sodium deoxycholate, 0.1% SDS, 1 mM EDTA, 1M NaCl, 1 to 4 M urea and 0.2 mM PMSF). The washed support-bound complexes can be collected and resuspended. As will be appreciated by one skilled in the art, the results obtained using the assay of the invention can be dependent on the quality of the antibody used.

Crosslinks can be reversed, for example, by incubating the complexes at about 4° C. to about 70° C. for about 45 min. Advantageously, the reversal is carried out in a buffer that is optimized to prevent unwanted RNA degradation or modification while maximizing reversal of the formaldehyde crosslink (e.g., 50 mM Tris-HCl, pH 7.0, 5 mM EDTA, 1% (w/v) SDS, 10 mM DTT). The pH below 7.6 reduces alkali digestion of RNA and the SDS prevents degradation by nucleases. Preferably, crosslinks resulting from the use of formaldehyde are reversed by incubating at about 70° C. for about 45 min. Resulting RNA can be extracted using any of a variety of techniques. For example, Trizol can be used. Any contaminating DNA can be removed by digestion with, for example, DNAase I, followed by removal of the DNase I.

The RNA immunoprecipitated in accordance with the invention can be analyzed using standard techniques, for example, by RT-PCR or RNA Invader Assay (Third Wave Technologies (Madison, Wis.) (Eis et al, Nature Biotech. 19:673 (2001))). Failure to detect an RT-PCR signal can result, for example, from incomplete reversal of crosslinks. The RT products or the RT-PCR products from an immunoprecipitation can be used to make cDNA probes for en masse analysis on gene expression chips (RIP on a Chip). Immunoprecipitated complexes can be used for further studies, as can the free proteins found in the precipitates following reversal.

In eukaryotic cells, post-transcriptional processing events such as pre-mRNA splicing, capping, polyadenylation, nuclear RNA transport, and translation represent critical steps in gene expression. These processes are highly regulated through the assembly and disassembly of specific RNP complexes. The use of formaldehyde for fixation of such RNP complexes provides a means for identifying interactions occurring under physiological conditions. Because of its high reactivity, formaldehyde rapidly reacts with free amines in proteins and nucleic acids producing protein-DNA, protein-RNA, and protein-protein crosslinks, which preserve the native architecture of cellular structures and thereby prevent the re-assortment of cellular components. The present assay can be used to identify in vivo interactions of proteins and RNA, including weak RNA-protein interactions. For instance, interactions between alternative splicing factors and RNA cis-acting elements, which control critical alternative splicing decisions, can be identified using this method. Equally, interactions between factors that modify messenger RNA stability and specific sites within these RNAs can be identified and mapped.

Certain aspects of the invention can be described in greater detail in the non-limiting Example that follows. (See also Niranjanakumari et al, Methods 26:182-190 (2002)).

### EXAMPLE

Experimental Details

In vivo formaldehyde fixation of cells: Actively dividing cultured human epithelial laryngeal carcinoma cells (HEp-2) growing as a monolayer (˜10⁶ cells), were first treated with trypsin, washed once with MEM medium containing 10% FBS (Invitrogen Corporation, CA), twice with 10 ml phosphate buffered saline (PBS) and resuspended in 10 ml of PBS. Formaldehyde (AR grade, Mallinckrodt) (from a 37% HCHO/10% methanol stock solution) was added to a final concentration of 1% (v/v, 0.36 M) and incubated at room temperature for 10 min with slow mixing. It is important to note that the buffers did not contain primary or secondary amines, since formaldehyde could crosslink these amines to proteins or nucleic acids. Crosslinking reactions were quenched by the addition of glycine (pH 7.0) to a final concentration of 0.25 M followed by incubation at room temperature for 5 min. The cells were harvested by centrifugation using a clinical centrifuge at 3,000 rpm (237×g) for 4 min followed by two washes with ice cold PBS. At this stage, the cell pellet could be stored at −80° C. until used. Fixed cells were resuspended in 2 ml of RIPA buffer (50 mM Tris-Cl, pH 7.5, 1% NP-40, 0.5% sodium deoxycholate, 0.05% SDS, 1 mM EDTA, 150 mM NaCl) containing protease inhibitors (complete, mini, EDTA-free protease inhibitor cocktail tablet, one tablet for 10 ml lysis buffer, Roche Molecular Biochemicals, IN).

Solubilization of crosslinked complexes by sonication: Mechanical shearing by sonication was used to solubilize RNPs. The cells were lysed routinely by 3 rounds of sonication, 20 s each in a Microson™ XL2007 ultrasonic homogenizer with a microprobe at an amplitude setting of 7 (output, 8-9 watts). Between each cycle, the samples were kept in an ice-water bath for at least 2 min. The efficiency of sonication was checked by examining 10 pi of the sample under a microscope; the sonicate was devoid of any intact cells. Insoluble material was removed by microcentrifugation at 14,000 rpm (16,000×g) for 10 min at 4° C.

Pre-clearing lysates: An aliquot of solubilized cell lysate (250 μl) was mixed with protein-A Sepharose beads (20 μl, packed volume) (Pharmacia, Piscataway, N.J.) along with non-specific competitor tRNA (final concentration of 100 μg/ml). This mixture was rotated for 1 hr at 4° C., followed by microcentrifugation at 4,000 rpm (1300×g) for 5 min. The pre-cleared supernatant was removed and used for the following immunoprecipitation step. An aliquot of this input was saved for RNA extraction.

Immunoprecipitation of crosslinked RNP complexes: Protein-A or protein-G Sepharose beads (20 μl, packed volume) were coated with the specific antibody (polyclonal sera) of interest for 2 hr at 4° C. followed by extensive washing with RIPA buffer containing protease inhibitors. Before immunoprecipitation, the beads were incubated for 10 min with 0.5 μl of RNasin (40 U/μl, Promega, Madison, Wis.). The pre-cleared lysate (250 μl) was diluted with RIPA buffer (250 μl), mixed with the antibody-coated beads and incubated with rotation at room temperature for 60-90 min. The beads were collected using a mini-centrifuge at 6,000 rpm for 45 s and the supernatant was saved for RNA extraction. The beads were washed 5 to 6 times with 1 ml of high stringency RIPA buffer (50 mM Tris-Cl, pH 7.5, 1% NP-40, 1% sodium deoxycholate, 0.1% SDS, 1 mM EDTA, 1 M NaCl, 1 to 4 M urea, and 0.2 mM PMSF) by 10 min rotation at room temperature. The beads containing the immunoprecipitated samples were collected and resuspended in 100 μl of 50 mM Tris-Cl, pH 7.0, 5 mM EDTA, 10 mM DTT and 1% SDS.

Reversal of crosslinks and RNA purification: Samples (resuspended beads) were incubated at 70° C. for 45 min to reverse the crosslinks. The RNA was extracted from these samples using Trizol according to manufacturer's protocol (Invitrogen Corporation, CA). Briefly, 100 μl of sample was mixed with 300 μl of Trizol followed by 80 μl of chloroform. The contents were mixed thoroughly and incubated at room temperature for 10 min. The organic and aqueous phases were separated by microcentrifugation at 12,000 rpm (11,750×g) for 10 min. The aqueous phase was collected and subjected to isopropanol precipitation in the presence of 1 μl of Glycoblue (Ambion Inc, Austin, Tex.) as a carrier. RNA precipitates were collected by microcentrifugation at 14,000 rpm (16,000×g) for 10 min, washed with 70% ethanol, air dried and resuspended in RNase free water. These samples were digested with DNase I (Buffer composition: 10 mM Tris-Cl, pH 7.5, 2.5 mM MgCl₂ and 0.1 mM CaCl₂) for 45 min at 37° C., followed by removal of the DNase I using DNase inactivating reagent (Ambion Inc, Austin, Tex.).

Analysis of immunoprecipitated RNA by RT-PCR: The RNA (1/10 of the total sample) purified from the previous step was used as a template to synthesize cDNA using random hexamers and MMLV reverse transcriptase (Invitrogen Corporation, CA) according to the manufacturer's protocol. RT reactions were carried out in 20 μl for 50 min at 37° C. followed by denaturation for 10 min at 70° C. PCR was performed in 50 μl volume containing 1×Taq buffer (10 mM Tris-Cl, pH 8.3, 50 mM KCl, 1.75 mM MgCl₂), 0.2 mM each dATP, dGTP, dCTP and dTTP, 10 pmole each specific primer, 1.25 U of Taq DNA polymerase and 2 μl of RT product. After an initial incubation at 94° C. for 4 min, the reaction mixtures were subjected to 25-35 cycles of amplification using the following sequence: 94° C. for 30 s, 60° C. for 36 s and 72° C. for 60 s, followed by a final extension step at 72° C. for 7 min. 15 μl of each reaction mixture was run on a 2% agarose gel and visualized by ethidium bromide staining.

Results

Development of the RIP Assay Using Hepatitis Delta Virus RNPs

Hepatitis delta virus (HDV) was used as a model system. The HDV genome is comprised of a 1700 nucleotide single-stranded circular RNA that is ˜70% self-complementary and as a result, forms a highly base-paired rod-like structure (Gerin et al in Fields Virology, Fourth editiion (Knipe and Howley, Eds.), pp. 3037-3050 (2001)). HDV replicates by a rolling circle mechanism. The incoming genome serves as a template for the production of complementary multimeric antigenomic RNAs. Nascent antigenomes use an intrinsic ribozyme activity to catalyze self-cleavage and self-ligation producing circular monomeric antigenomic RNAs (Been and Wickham, Eur. J. Biochem. 247:741-753 (1997)). Through a similar rolling circle mechanism, these antigenomes serve as templates for the production of additional genomic RNAs. The genomic RNA also serves as a template for the production of a mRNA that encodes the only HDV protein, hepatitis delta antigen (HDAg), which is absolutely essential for HDV replication. Thus, during its life cycle, the virus produces three types of RNAs: genomic and antigenomic circular RNAs and the HDAg mRNA. The genomic RNA is abundant (˜300,000 copies per cell) compared to antigenomic RNA (˜50,000 copies) and mRNA (˜600 copies) (Taylor, Annu. Rev. Microbiol. 46:253-276 (1992)). HDAg binds to both genomic and antigenomic RNA with high affinity but not to its own mRNA. Multiple features of the HDV life cycle including abundant RNAs, high affinity of HDAg for the HDV RNAs, and the availability of reagents, such as good immunoprecipitating anti-HDAg antibodies (Brazas and Ganem, Science 274:741-753 (1997)) and various HDV mutants (Lazinski and Taylor, J. Virol. 68:2879-2888 (1994)), make this an excellent model system for the RIP assay.

Specific crosslinking of HDAg to HDV RNA. To study the in vivo binding of HDAg to HDV RNA, human epithelial laryngeal carcinoma cells (HEp-2) were transfected with a plasmid expression vector that expresses a 1.2×, linear HDV genomic RNA from the SV40 late promoter. The HDV genomic ribozyme is duplicated at each end of this transcribed RNA. These ribozymes cleave the genomic RNA at both ends generating a linear, monomeric genomic RNA, which then circularizes by ligation. This circular genomic RNA can then begin the replication process. This plasmid also expresses HDAg which is required to initiate HDV replication. Four days post-transfection, cells were harvested and crosslinked as described above.

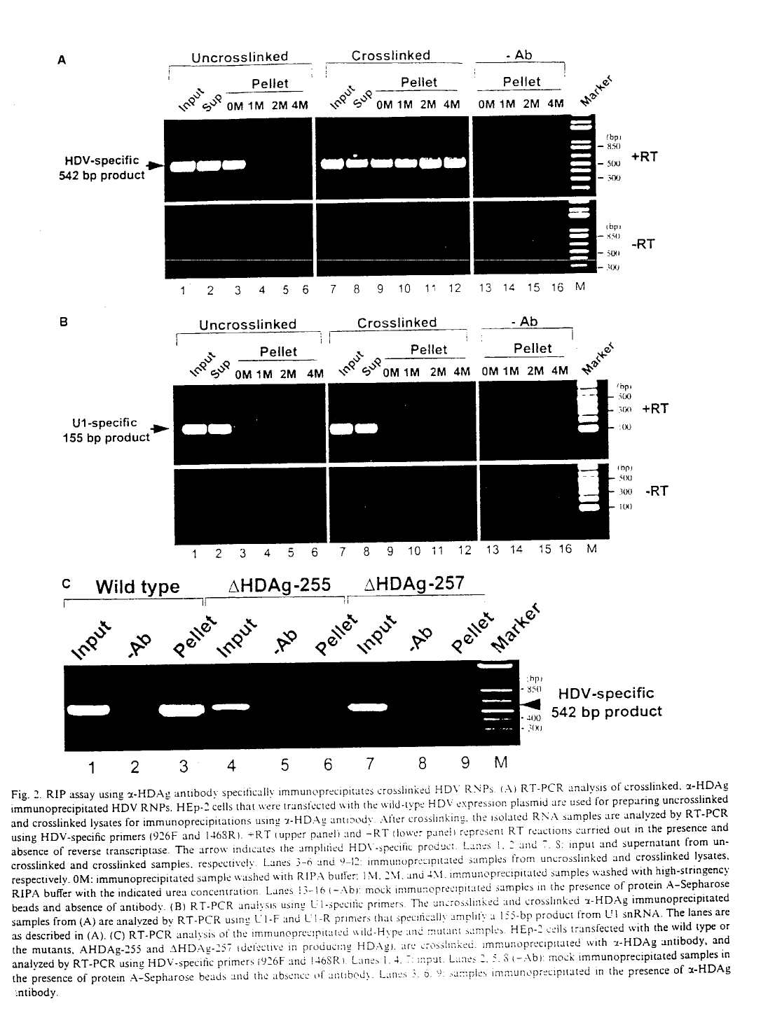

Initially 1% formaldehyde was used for 10 min to crosslink HDAg to HDV RNAs in HEp-2 cells that were transfected with the HDV expression plasmid. Upon optimization of formaldehyde concentrations, crosslinking with 0.4 to 0.6% formaldehyde for 10 min was found to be optimal. Most of the crosslinking experiments were performed using 0.6% formaldehyde for 10 min. Following immunoprecipitation using α-HDAg antibody, the crosslinks were reversed at 70° C. for 45 min. These reversal conditions were selected based on a time course from 5 min to 6 hours at 70° C. RNAs isolated from reversed samples were analyzed by RT-PCR using random hexamers for the reverse transcription, and HDV-specific primers (926F, 5′-AAACCTGTGAGTGGAAACCCG and 1468R, 5′-ATAGAGGACGAAAATCCCTGGC) for the PCR amplification. Primer, 926F is forward primer, while 1468R is a reverse primer and their sequences correspond to nucleotides 926-946 and 1468-1447 respectively of the genomic strand of HDV RNA; the PCR reaction using these primers results in the production of a 542 bp product. As shown in FIG. 2A, the 542 bp HDV-specific product was evident in the immunoprecipitated pellets suggesting the crosslinking of HDV RNA to HDAg (lanes 9-12). No RT-PCR product was detected in samples that were not subjected to reversal conditions.

HDAg is known to associate tightly with HDV RNA genome (Gerin et al in Fields Virology, Fourth editiion (Knipe and Howley, Eds.), pp. 3037-3050 (2001)). To distinguish the binding of HDAg to HDV RNA in uncrosslinked and crosslinked cells, the immunoprecipitated samples were washed with high stringency RIPA buffer containing 1 M, 2 M or 4 M urea. The results are summarized in FIG. 2A. In the uncrosslinked samples, the 542 bp product was detected in the immunoprecipitated samples that were not washed with urea buffer (lane 3), but the signal is absent in samples that were washed with high stringency buffers (lanes 4-6). The presence of the 542 bp RT-PCR product in the crosslinked, immunoprecipitated samples (lanes 9-12) indicates that formaldehyde covalently crosslinked HDAg to the HDV RNA. These crosslinks are shown to be resistant even to a 4 M urea wash (lane 12). This indicates that in the absence of formaldehyde, the HDAg-RNA interactions are disrupted under high stringency conditions, whereas these interactions are retained upon formaldehyde crosslinking. The RT-PCR signal in the immunoprecipitates is dependent on the presence of antibody as seen by the absence of signal in the mock precipitation (lanes 13-16). Linearity in the PCR amplification was further confirmed by repeating the PCR using serial dilutions of the RT product. The presence of HDV-specific product was readily visible as early as 24 cycles of amplification with 1:10 diluted RT product in crosslinked, immunoprecipitated samples. On the other hand, this product was completely absent even after 28 cycles in undiluted RT product in uncrosslinked, immunoprecipitated samples washed with urea buffer (data not shown). These results demonstrated the specific crosslinking of HDV RNA to HDAg.

HDAg antibodies specifically immunoprecipitates HDV RNA-protein complexes. To assess the specificity of the RIP assay for the HDV RNP, a test was made for the presence of a non-specific RNA in the HDV-RNP immunoprecipitates. U1 snRNA was chosen as a negative control for this experiment because like the HDV RNAs, U1 snRNA is a highly abundant nuclear RNA (Baserga et al in The RNA World (Gesteland and Atkins, Eds.), pp. 359-381 (1993)). RNA isolated from crosslinked, anti-HDAg immunoprecipitated complexes was analyzed by RT-PCR using U1 snRNA specific primers, U1-F (5′-CTTACCTGGCAGGGGAGATAC, binds to nt 4-24 region of U1 snRNA) and U1-R (5′-GMAGCGCGMCGCAGTC, binds to nt 159-142 region of U1 snRNA). This primer pair resulted in the amplification of a 155 bp U1 snRNA-specific product. As indicated in FIG. 2B, the U1 snRNA specific product was evident in the input and supernatant fractions in both uncrosslinked and crosslinked samples (lanes 1-2 and 7-8). This product was faintly visible in the uncrosslinked immunoprecipitated samples that were washed with buffers containing 0 or 1 M urea (lanes 3-4), but at higher urea concentration no signal was detected in either uncrosslinked or crosslinked samples (lanes 5-6 and 11-12). These results are in marked contrast to results from the HDV-specific RT-PCR, where HDV RNA can be detected with washes all the way up to 4 M urea (FIG. 2A). As expected, no product was detected when reverse transcriptase was omitted. The almost complete absence of U1 snRNA specific product in the crosslinked immunoprecipitated samples demonstrated the specificity of RIP assay for the HDV RNP.

To test whether the results obtained above were HDAg dependent, immunoprecipitations were performed using crosslinked lysates from cells transfected with the wild type HDV expression vector as well as variants, HDAg-255 and HDAg-257. The wild type plasmid expresses both genomic and antigenomic strands of HDV, while mutants HDAg-255 and HDAg-257 express only the antigenomic or the genomic strand, respectively, of the HDV RNA genome. Both the variants contain a 2 base pair insertion in the HDAg ORF. This frameshift mutation prevents the expression of functional HDAg in these cells and prevents the replication of the HDV RNA (Lazinski and Taylor, J. Virol. 68:2879-2888 (1994)). RNA isolated from the crosslinked, immunoprecipitated samples was analyzed by RT-PCR using HDV-specific primers (926F and 1468R). As shown in FIG. 2C, the HDV-specific 542 bp product was clearly detectable in the input samples from the wild type and both the HDAg mutants (lanes 1, 4 and 7). This product was evident in the crosslinked, α-HDAg immunoprecipitated samples from the wild type (lane 3) but not in the crosslinked, immunoprecipitated samples from HDAg mutants (lanes 6 and 9) showing that, as expected, the signal observed in the RIP assay is HDAg-dependent. These data indicate that precipitation of the HDV genomic and antigenomic RNAs is fully dependent on the presence of functional HDAg.

Immunoprecipitation of crosslinked HDV lysates with Sm antibodies. The specificity of the RIP assay for the HDV RNP was further tested using unrelated antibodies. In this case, Sm-specific antibodies were used for the immunoprecipitation step with the crosslinked HDV lysates. Sm antibodies are detected in patients with the autoimmune disease systemic lupus erythematosus (SLE) (Lerner and Steitz, Proc. Natl. Acad. Sci. USA 76:5495-5496 (1979), Wilusz and Keene, J. Biol. Chem. 261:5467-5472 (1986)), and they recognize Sm antigens, components of the RNA splicing machinery. Two different antisera were utilized for these experiments. The first, AW, contains Sm-specific antibodies and hence served as positive sera. The second, JM, does not possess any anti-Sm specific antibodies and was used as control sera when testing for Sm antigen-specific RNAs. To determine whether Sm antibodies immunoprecipitate any crosslinked HDAg-HDV RNA complexes, the AW and JM antisera immunoprecipitated samples were analyzed by RT-PCR using HDV-specific primers (926F and 1468R). As indicated in FIG. 3, both AW and JM antibodies failed to precipitate any HDAg-RNA complex as evidenced by the absence of 542 bp HDV-specific product even after 32 cycles of amplification (lanes 9 and 11). The presence of this product is easily detected in the input as well as supernatant fractions (lanes 7, 8 and 10). These results further confirm the specificity of the RIP assay for the HDV RNP when HDAg-specific antibodies are used.

Application of the RIP Assay to U1 snRNP.

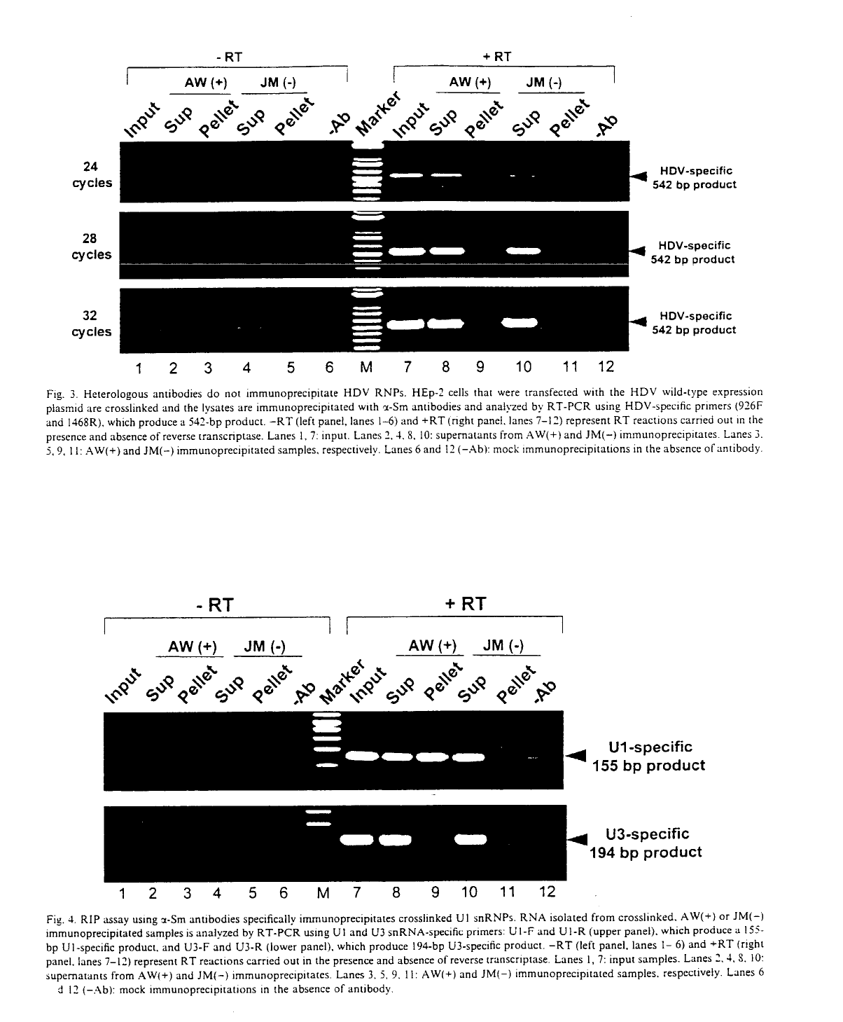

To verify that the Sm antibodies did indeed recognize and immunoprecipitate the appropriate Sm antigens and their associated RNAs, the Sm immunoprecipitates were tested for the presence of Sm associated RNAs by RT-PCR. Sm antigens consist of the U1, U2, U4/U6 and U5 snRNPs, which are named after their specific snRNA components (U1, U2, U4/U5 and U5). Immunoprecipitations were performed using AW and JM antisera with crosslinked samples prepared from HEp-2 cells that were replicating wild type HDV. RT-PCR was performed using U1 snRNA specific primers (U 1-F and U1-R) and the results are shown in FIG. 4 (upper panel). The antibody AW specifically immunoprecipitated U1 snRNA as evidenced by the presence of the expected 155 bp product, which is absent in control JM immunoprecipitated samples (compare lane 9 to lane 11, upper panel). The U3 snRNP does not contain Sm proteins, and therefore, the U3 RNA should not be detected in the AW immunoprecipitates and was used as an additional negative control. The U3 snRNA-specific primers (U3-F, 5′-CAGGGATCATTTCTATAGTGTG, binds to nt 13-35, and U3-R, 5′-AGACCGCGTTCTCTCCCTCTCA, binds to nt 210-189 of U3 snRNA) produced the expected 194 bp U3 snRNA specific product in the input and supernatant samples (lanes 7, 8 and 10, lower panel) but was completely absent in both AW and JM immunoprecipitated samples (lanes 9 and 11, lower panel). This experiment demonstrates that the Sm antibodies present in the AW sera were fully capable of immunoprecipitating the crosslinked Sm antigens, in particular the U1 snRNP, thus extending the usefulness of the RIP assay to a second RNP, namely U1 snRNP.

Application of the RIP assay to RNPs that are much less abundant than HDV RNP and U1 snRNP (e.g., an unspliced precursor to fibroblast growth factor-2 messenger RNA) has been attempted, however, not yet successfully. The reasons for this could be many. It may reflect an inherent limitation in the sensitivity of the assay, due, for instance, to incomplete reversal of crosslinks, or it may be an indication of proclivities of the antibodies that were used to evaluate the rare nuclear RNAs.

All documents cited above are hereby incorporated in their entirety by reference.

What is claimed is:

1. A method of identifying an RNA that binds to a protein comprising:
i) contacting a cell comprising a complex that comprises said protein and said RNA with a reversible crosslinking agent under conditions such that said complex is crosslinked,
ii) lysing said cell,
iii) isolating said crosslinked complex from said lysate resulting from step (ii),
iv) treating said isolated crosslinked complex resulting from step (iii) under conditions such that said crosslinks are reversed and said RNA dissociates from said protein, and
v) identifying said dissociated RNA or protein, or both.

2. The method according to claim 1 wherein, in step (iii), said crosslinked complex is isolated by contacting said lysate with an antibody, or binding fragment thereof, specific for said protein under conditions such that said antibody binds to said crosslinked complex, and isolating complex-bound antibody from said lysate.

3. The method according to claim 1 wherein said cell is a eukaryotic cell.

4. The method according to claim 1 wherein said cell is a cultured cell.

5. The method according to claim 1 wherein said crosslinking agent is formaldehyde.

6. The method according to claim 5 wherein said cell is contacted with formaldehyde at a final concentration of 0.1% (v/v) to 10% (v/v).

7. The method according to claim 6 wherein said cell is contacted with formaldehyde at a final concentration of about 1% (v/v).

8. The method according to claim 1 wherein, in step (i), said crosslinking agent is not present in a buffer comprising a primary or secondary amine.

9. The method according to claim 1 wherein, in step (i), said crosslinking agent is present in a buffer selected from the group consisting of a phosphate, Hepes and triethanolamine buffer.

10. The method according to claim 1 wherein said method further comprises contacting the composition resulting from step (i) with an inhibitor of said crosslinking agent.

11. The method according to claim 10 wherein said inhibitor is a compound with a primary amine.

12. The method according to claim 11 wherein said inhibitor is glycine.

13. The method according to claim 1 wherein, in step (ii), said lysing is effected by mechanical shearing.

14. The method according to claim 1 wherein said method further comprises, after step (ii), removing non-specific aggregates.

15. The method according to claim 1 wherein, in step (iv), said crosslinks are reversed by incubating said isolated crosslinked complex at about 4° C. to about 70° C. for about 45 min.

16. The method according to claim 1 wherein, in step (iv), said treatment is carried out in a buffer that inhibits RNA degradation.

17. The method according to claim 1 wherein said crosslinking agent is formaldehyde and, in step (iv), said crosslinks are reversed by incubating said isolated crosslinked complex at about 70° C. for about 45 min.

18. A method of mapping a specific binding site of a protein to an RNA comprising:
i) contacting a cell comprising a complex that comprises said protein and said RNA with a reversible crosslinking agent under conditions such that said complex is crosslinked,
ii) lysing said cell,
iii) treating said crosslinked complex so that fragments of said RNA crosslinked to said protein are formed,
iv) isolating from said lysate said RNA fragments crosslinked to said protein,
v) treating the composition resulting from step (iv) under conditions such that said crosslinks are reversed and said RNA fragments dissociate from said protein, and
vi) identifying said RNA fragments and thereby mapping the site of binding of said protein to said RNA.

19. The method according to claim 18 wherein said RNA fragments are about 200 to 500 nucleotides long.

20. The method according to claim 18 wherein, said treatment of step (iii) is effected using sonication or limited nuclease digestion.
